(19) United States
(12) Patent Application Publication
(10) Pub. No.: US 2004/0065334 A1
(43) Pub. Date: Apr. 8, 2004

(54) Hand acupuncture

(76) Inventor: Tae Woo Yoo, Seoul (KR)

Correspondence Address: Richard M. Goldberg, Suite 419, 25 East Salem Street, Hackensack, NJ, 07601, US

(21) Appl. No.: 10/614,143

(22) Filed: Jul. 7, 2003

(30) Foreign Application Priority Data
Oct. 8, 2002 (KR) 2002-29941

(51) Int. Cl. A61B 17/04

(52) U.S. Cl. 128/907

(57) ABSTRACT

A hand acupuncture needle assembly according to the present invention comprises an acupuncture needle including a body, and a fine point at a bottom thereof, and a tube type grip element extending over a portion of the acupuncture needle, and at least a substantial length of the tube type grip element being compressed such that an outer surface of the acupuncture needle is fixedly connected with an inner surface of the at least a substantial length of the grip element.

### BACKGROUND OF THE INVENTION

The present invention relates to a hand acupuncture needle assembly, and more particularly, a hand acupuncture needle assembly comprising an acupuncture needle including an upper grip part, a body and a fine point at the bottom. The grip part is inserted into the upper part of a tube type grip element and compressing the grip element to be bound to the upper grip part of the acupuncture needle. The hand acupuncture needle assembly of the present invention can be simply manufactured with a low defective or inferiority rate. Thus, the hand acupuncture needle assembly can be produced with a reduced production cost and provide an economic effect.

Generally, a hand acupuncture needle has a diameter of about 0.2 mm and a length of about 1.5 cm. It is difficult to apply such acupuncture needle by grasping with the hand because such acupuncture needle is too small and thin. In order to solve this problem, the upper part of the acupuncture is made irregular and inserted into a grip element which is coiled around it. However, there are some problems in that the manufacture of the irregular portion and the grip element is complicated, and thereby causes a high defective rate and an increased production cost. Therefore, there has been a need for new acupuncture needle assembly which is readily manufactured and conveniently used.

Korean Utility Model Application No. 2002-25613, which was previously filed by the present applicant, relates to a hand acupuncture needle assembly which comprises a grip part, a body and a fine bottom portion. The grip part of this hand acupuncture needle assembly is made by inserting the upper part of the needle into a tube type grip element and compressing the grip element to be bound to the top point of the acupuncture needle. Since the hand acupuncture needle assembly is made by inserting the upper part of the acupuncture needle into the grip element and compressing the grip element, the manufacture is simple and thereby the defective rate is low. It is effective to cut down the production cost. When operating, a user inserts the hand acupuncture into the affected part and rotates it by grasping the grip part or taps it to provide stimulation. However, since the grip element is bound to only the top point of the upper part of the needle, the bound site makes the rotation of the hand acupuncture needle assembly difficult, and thus provides a reduced effect of operation. Also, since the grip element is bound to the top point of the upper part of the needle, and the remaining part of the grip element remains unbound, the unbound part of the grip element can be shaken. This causes difficulty for providing accurate operation on the acupuncture point.

### SUMMARY OF THE INVENTION

The present invention was designed to solve the aforementioned problems.

An object of the present invention is to provide an acupuncture needle assembly which can be easily manufactured and conveniently used. Since it is manufactured by combining the upper part of the acupuncture needle with a grip element, the hand acupuncture needle assembly of the present invention can be produced with a low cost. The hand acupuncture needle assembly according to the present invention comprises an acupuncture needle including a body, and a fine point at a bottom thereof, and a tube type grip element extending over a portion of the acupuncture needle, and at least a substantial length of the tube type grip element being compressed such that an outer surface of the acupuncture needle is fixedly connected with an inner surface of the at least a substantial length of the grip element. When the grip element is compressed to the acupuncture needle, the inner diameter of the tube type grip element is decreased, and thereby the grip element is extended in upper and lower directions to provide adhered portions.

The hand acupuncture needle assembly of the present invention can be simply manufactured with a low defective rate. Thus, the hand acupuncture needle assembly can be produced with a reduced production cost and provide an economic effect.

When operating, a user inserts a hand acupuncture needle into the affected part and rotates it by taking the grip part or taps it to provide stimulation. Since the grip element of the present hand acupuncture needle assembly has a smooth surface, the operation is convenient. The hand acupuncture of the present invention can be operated by grasping it directly as well as by using an acupuncture tube.

### BRIEF DESCRIPTION OF THE DRAWINGS

The other objects and features of the present invention will be hereinafter explained in detail with reference to the accompanying drawings.

FIG. 1 is an exploded perspective view showing the separate, pre-manufacturing state of the present invention.

FIG. 2 is a perspective view showing the manufactured state of the present invention.

FIG. 3 is a combined cross-sectional view showing the pre-manufacturing state of the present invention.

FIG. 4 is a combined cross-sectional view showing the manufactured state of the present invention.

FIG. 5 is an enlarged cross-sectional view of part A of FIG. 3.

FIG. 6 is an enlarged cross-sectional view of part B of FIG. 4.

FIG. 7 is an illustrated perspective view showing an operation state of the present invention using an acupuncture tube.

FIG. 8 is an illustrated perspective view showing operation of the present invention by hand.

FIG. 9 is an enlarged cross-sectional view showing a pre-manufacturing state of the present invention.

FIG. 10 is an enlarged cross-sectional view showing a manufactured state of the present invention.

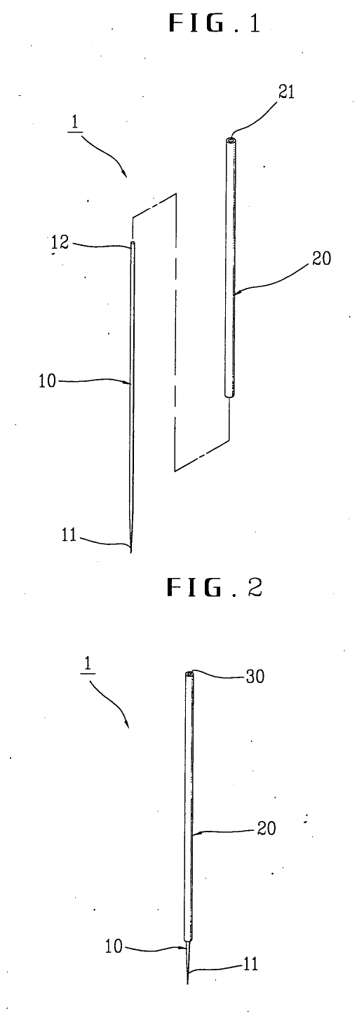

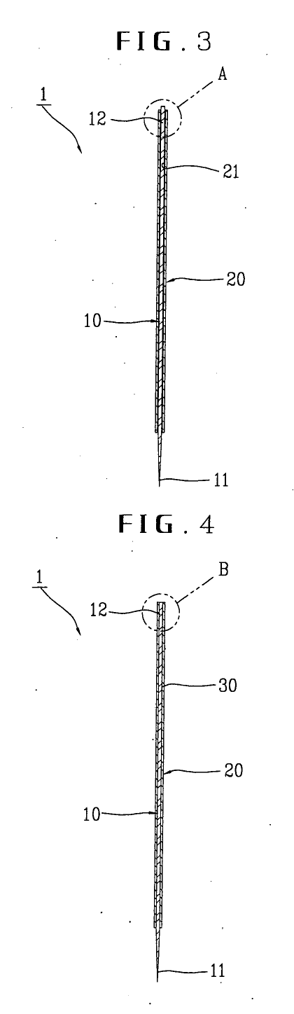

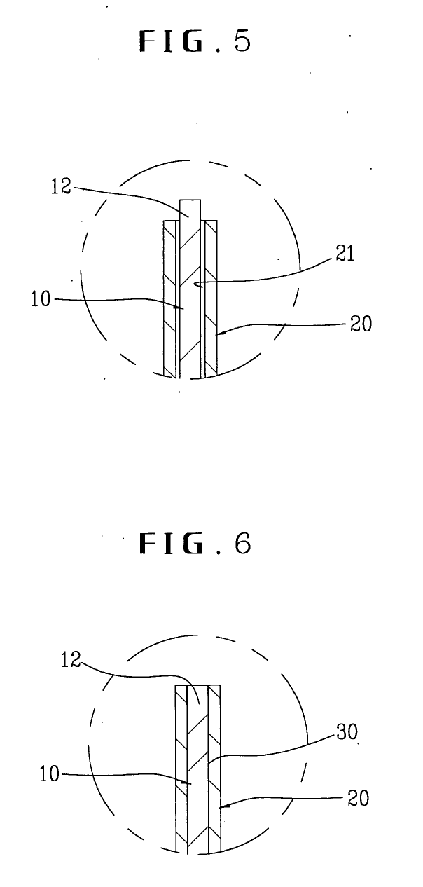

### DETAILED DESCRIPTION OF THE INVENTION

A hand acupuncture needle assembly 1 according to the present invention comprises a needle having an upper grip part 12, a body 10 and a fine point 11 at the bottom. The upper grip part 12 of the acupuncture needle is inserted into a tube type grip element 20 and the grip element 20 is compressed firmly to be bound to upper grip part 12, which results in the formation of the compressed adhered portions 30 between the outer surface of upper grip part 12 and the inner surface of the upper part 21 of grip element 20. Since, as shown in the drawings, grip element 20 is compressed along at least substantially its entire length, a strong connection is made between the acupuncture needle and the grip element 20.

Numerals 100 and 200 refer to the skin and an acupuncture tube, respectively.

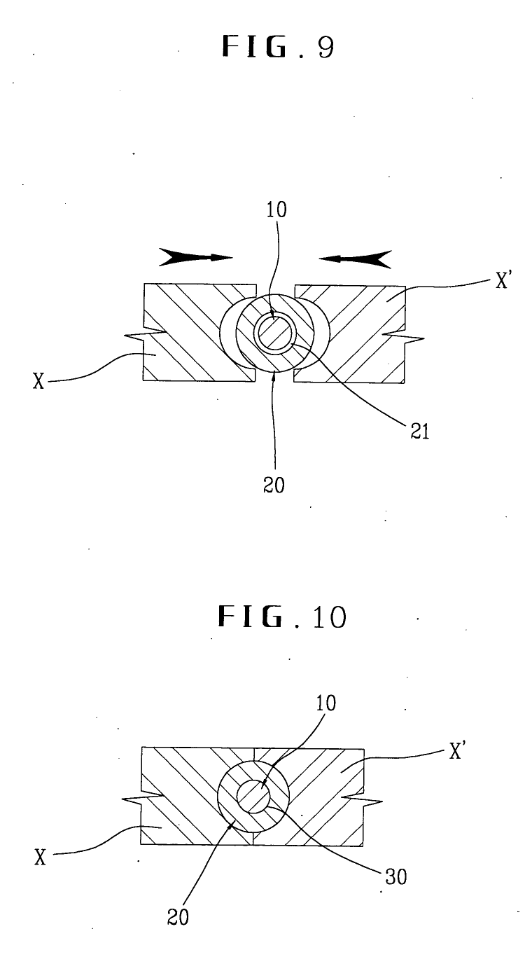

As discussed above, hand acupuncture needle assembly 1 of the present invention comprises an acupuncture needle having a fine point 11 at the bottom and a grip part 12 at the upper end of the body 10, and grip part 12 is inserted into upper part 21 of tube type grip element 20. As illustrated in FIG. 9, a pair of compressor elements X, X′ are used to compress the entire tube type grip element 20. When compressed, the inner diameter of the tube type grip element 20 is decreased, and thereby, grip element 20 is extended to upper and lower directions to cause the formation of adhered portions 30 between the outer surface of the acupuncture needle and the inner surface of the grip element 20.

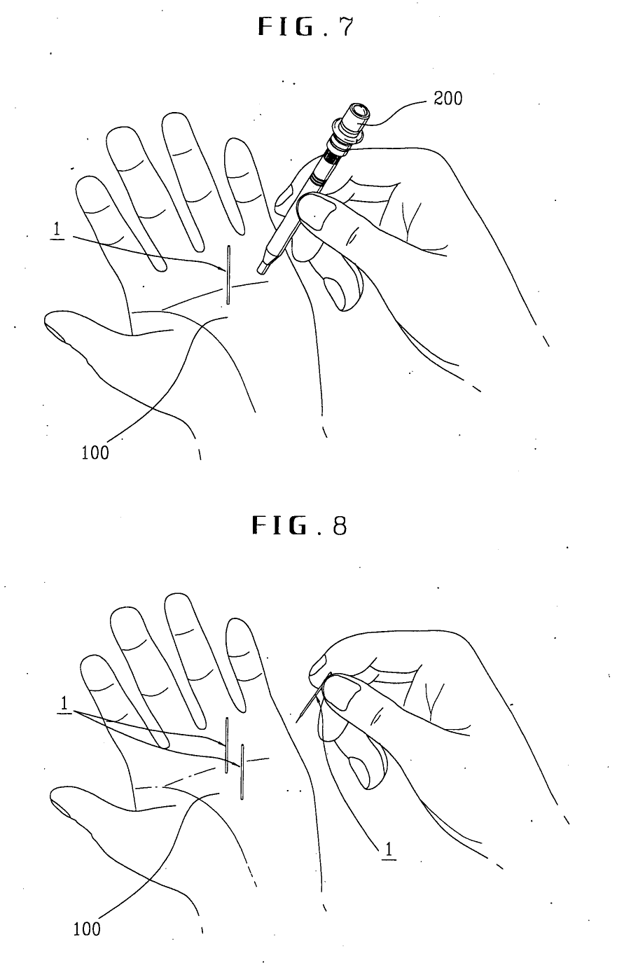

As illustrated In FIG. 7, hand acupuncture needle assembly 1 may be operated onto skin 100 by inserting it into acupuncture tube 200. It can be also used directly by grasping grip element 20. After applying acupuncture to skin 100, grip element 20 of hand acupuncture needle assembly 1 is rotated or tapped to provide stimulation.

As mentioned above, a pair of compressor elements X, X′ are used to compress the entire tube type grip element 20 as illustrated in FIG. 9. When compressed, the inner diameter of tube type grip element 20 is decreased, and thereby, grip element 20 is extended in upper and lower directions to cause the formation of adhered portions 30 between the outer surface of the acupuncture needle and the inner surface of grip element 20. Since the manufacturing process of the present hand acupuncture needle assembly 1 is simple, hand acupuncture needle assembly 1 is produced with a low defective rate and with less cost. When operating onto skin 100, it is easy to rotate or tap grip part 12 because grip element 20 has a smooth surface. As illustrated in FIG. 7, hand acupuncture needle assembly 1 can be operated on skin 100 by inserting it into acupuncture tube 200, positioning tube 200 on the skin and allowing a weight to fall down. In this case, hand acupuncture needle assembly 1 is operated by the dropping power of the weight. As illustrated in FIG. 8, hand acupuncture needle assembly 1 can be also operated directly by grasping grip element 20. As described above, hand acupuncture needle assembly 1 of the present invention can be readily manufactured and conveniently used.

What is claimed is:

1. An acupuncture needle assembly comprising:
an acupuncture needle including:
a body, and
a fine point at a bottom thereof, and
a tube type grip element extending over a portion of the acupuncture needle, and
at least a substantial length of the tube type grip element being compressed such that an outer surface of the acupuncture needle is fixedly connected with an inner surface of the at least a substantial length of the grip element.

2. An acupuncture needle assembly according to claim 1, wherein said grip element has a generally cylindrical outer surface.

3. An acupuncture needle assembly according to claim 1, wherein said tube type grip element is compressed along an entire length thereof around said acupuncture needle.
